(19) United States
(12) Patent Application Publication
(10) Pub. No.: US 2005/0070944 A1
(43) Pub. Date: Mar. 31, 2005

(54) Method and apparatus for isolating cell nuclei from biopsy obtained tissue

(75) Inventors: Mark R. Holl, Shoreline, WA (US); Andrew K. Miller, Quilcene, WA (US); Matthew L. Stanton, Mercer Island, WA (US); Carissa A. Sanchez, Seattle, WA (US); Thomas G. Paulson, Shoreline, WA (US); Brian J. Reid, Seattle, WA (US); Deirdre R. Meldrum, Mercer Island, WA (US)

Correspondence Address: Delbert J. Barnard, Esq., P.O. Box 58888, Seattle, WA, 98138-1888, US

(73) Assignees: Fred Hutchison Cancer Research Center; University of Washington

(21) Appl. No.: 10/847,950

(22) Filed: May 14, 2004

Related U.S. Application Data
(60) Provisional application No. 60/471,164, filed on May 15, 2003.

(51) Int. Cl. A61B 17/14

(52) U.S. Cl. 606/180

(57) ABSTRACT

A rotor (14) is located within a rotor chamber (12) formed in a housing (24). A drive shaft (16) extends from a motor and couples to the drive shaft interconnect (46) at the center of the rotor (14). One side of the rotor (14) is provided with circumferentially spaced apart radial rows of mincing edges (20). These edges (20) are situated on concentric circles or on a spiral. They are elongated circumferentially and may be situated substantially on an arc and substantially on an arc or on a chord. During use, the rotor (14) is rotated after biopsy obtained tissue (10) has been placed in the rotor chamber (12) on the side of the rotor (14) that includes the mincing edges (20). A fluid is delivered into and out from the rotor chamber (12) as the rotor (14) is being rotated to mince the biopsy, lyse the cells, and release the cell nuclei from the obtained tissue. Cell nuclei are expelled from the chamber (12) and are moved through a filter (58). The cell nuclei are collected and then delivered to a flow cytometry apparatus to be analyzed.

### RELATED APPLICATIONS

This application claims priority to Provisional Application Ser. No. 60/471,164, filed May 15, 2003, and entitled Tissue Disaggregator For Small Biopsies.

The contents of Provisional Application Ser. No. 60/471,164 are hereby incorporated herein by this specific reference.

### STATEMENT OF INTEREST

The invention disclosed and claimed herein was developed under NIH/NHGRI Grant No. P50 HG002360-02, “CEGSTech: Integrated Biologically-Active Microsystems,” and NIH Grant No. P01 CA91955, “Barrett's Esophagus: Predictors of Progression.” The United States government may have rights to the invention.

### TECHNICAL FIELD

The present invention relates to an apparatus and method for disaggregating body tissue, obtained by a biopsy, to free and isolate cell nuclei. It also relates to such an apparatus and method for staining, filtering and isolating the cell nuclei in a format allowing it be directly analyzed by flow cytometry.

### BACKGROUND OF THE INVENTION

There are diseases for which diagnostic information can be obtained through use of flow cytometry, an analysis technique that requires isolation of cell nuclei from biopsy obtained tissue. Current techniques for isolating cell nuclei include manual disaggregation of the tissue, typically by use of scalpel blades to mince and tease the tissue apart. This occurs in the presence of a non-ionic detergent solution which keeps the tissue from drying out as well as permeabilizing the cell, but not the nuclear, membrane. The disaggregated tissue is then repeatedly drawn into and expelled from a 1000 μl pipet tip. This further disaggregates the tissue and begins releasing nuclei from the cells in the tissue. Finally, the tissue suspension is repeatedly forced through a 25-gauge needle which generates sufficient hydrodynamic forces combined with the action of a lytic reagent to enucleate the majority of the cells, resulting in a mixture of nuclei (typical size 10 μm) and cellular debris. This mixture is then forced through a stainless steel mesh screen that traps the cellular and tissue debris and allows single nuclei to pass through. Isolated nuclei are then resuspended in a solution containing the DNA staining dye 4′, 6-diamidino-2-phenylindole (DAPI), which allows quantitation of DNA content.

The manual disaggregation of tissue and isolation of cell nuclei requires extensive labor and is technically difficult. As a result, flow cytometry is not performed in most clinical laboratories. There is a need for a way of facilitating the isolation of cell nuclei so that flow cytometry can be performed in nearly all clinical laboratories. Fulfilling this need would facilitate a more rapid translation of basic research findings to improved patient care. It is the primary object of the present invention to fulfill this need by providing an apparatus and method that will automatically disaggregate biopsy tissue and filter and stain and isolate cell nuclei.

### BRIEF SUMMARY OF THE INVENTION

A mechanical tissue disaggregator constructed according to the present invention is basically characterized by a housing including a rotor chamber and a rotor in the rotor chamber. The rotor includes a drive-motor interconnect and tissue mincing edges. The housing includes a fluid inlet leading into the rotor chamber and an outlet from the rotor chamber. In preferred form, the chamber drive-motor interconnect is at the center of the rotor and is adapted to receive a drive shaft that is axially alignable with the drive-motor interconnect.

According to an aspect of the invention, the housing includes a reagent/stain inlet passageway leading into the rotor chamber. It also includes an outlet passageway leading out from the rotor chamber to an outlet port.

In the preferred embodiment, the mincing edges stand out on a side of the rotor and are spaced apart both radially and circumferentially. The mincing edges may be arranged in radial rows on concentric circles or on a spiral. In one embodiment, filtration channels extend radially on the same side of the rotor as the mincing edges. These filtration channels extend radially outwardly from the mincing edges, to the periphery of the rotor.

According to an aspect of the invention, the housing and rotor may form a disposable assembly. The housing, the rotor and the tissue mincing edges may all be formed from a single piece of plastic material, e.g., a castable urethane material, or a embossed acrylic.

According to another aspect of the invention, the housing may include a side member that is hinge connected to another portion of the housing. This side member has a closed position, in which it forms a side closure for the rotor chamber and is outwardly adjacent the side of the rotor that includes the tissue mincing edges, and an open position in which it is swung out from the rotor and exposes the rotor and the rotor chamber.

The method aspect of the invention comprises providing a housing that includes a rotor chamber and a rotor in the rotor chamber, and proving the rotor with mincing edges. A biopsy is conducted to obtain tissue. The tissue is placed in the rotor chamber contiguous with the mincing edges. A liquid reagent is directed into and through the rotor chamber. While the rotor is rotated, causing the mincing edges to tease the tissue apart.

In the preferred embodiment, the housing and rotor are a part of a disposable assembly. They are placed on a holder in a position to receive a drive shaft that couples with a drive-motor interconnect at the center of the motor. The drive motor is operated to rotate the shaft and the shaft in turn rotates the rotor.

According to another aspect of the invention, a plurality of cassette stations are provided. Each station includes connectors for connecting a cassette at such station to conduits which extend to and from the connectors, enabling a plurality of cassettes to be used in a sequence or at the same time.

Other objects, advantages, and features of the invention will become apparent. From the description of the best mode set forth below, from the drawings, from the claims and from the principles that are embodied in the specific structure that are illustrated and described.

### BRIEF DESCRIPTION OF THE SEVERAL VIEWS OF THE DRAWING

Like reference numerals are used to designate like parts throughout the several views of the drawing, and:

FIG. 1 is a flow diagram of a process based on apparatus and method aspects of the present invention;

FIG. 2 is an exploded view of an apparatus embodiment of the present invention, showing some parts in elevation and other parts in section;

FIG. 3 is an enlarged scale sectional view of the rotor and rotor chamber portions of FIG. 2;

FIG. 4 is a plan view of a basic cassette provided with a single reagent inlet, a disaggregator rotor, a filter and a product outlet;

FIG. 5 is a view like FIG. 4 but of a modified cassette that includes passive mixing;

FIG. 6 is a view like FIG. 4 but of a cassette that includes an active sonic mixer;

FIG. 7 is a longitudinal sectional view taken substantially along line 7-7 of FIG. 6;

FIG. 8 is a view like FIG. 4 but of an embodiment in which the rotor includes filter channels;

FIG. 9 is a pictorial view of the rotor of FIG. 8, looking towards a side that includes mincing edges and filter channels;

FIG. 10 is a plan view of the rotor shown by FIGS. 8 and 9;

FIG. 11 is an enlarged scale fragmentary view of a group of mincing blades;

FIG. 12 is an enlarged scale pictorial view of a single mincing blade, showing how it projects out from the face of the rotor;

FIG. 13 is a fragmentary sectional view showing a mincing blade on the rotor moving relative to posts on the bottom of the rotor cavity in the cassette;

FIG. 14 is a diagram of the steps of a method of the invention starting with the placement of biopsy tissue in the rotor chamber of the cassette;

FIG. 15 is a diagram like FIG. 14 illustrating the steps involved in removing the cassette and product from the system;

FIG. 16 is a diagram of a system that includes multiple processing stations;

FIG. 17 is an exploded pictorial view of different parts of a cassette, such view showing a disaggregation rotor spaced from a rotor chamber and top and bottom closures for the chamber; and

FIG. 18 is a view of the component shown by FIG. 17 with the top closure shown connected to the remaining portion of the cassette housing by a hinge, allowing the closure to be moved between open and closed positions.

### DETAILED DESCRIPTION OF PREFERRED EMBODIMENTS

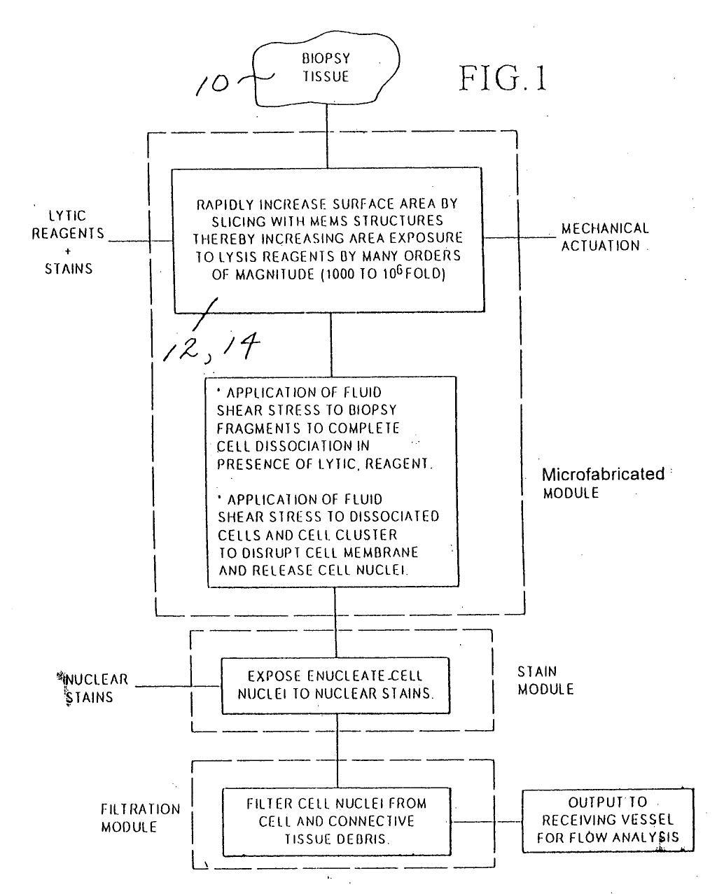

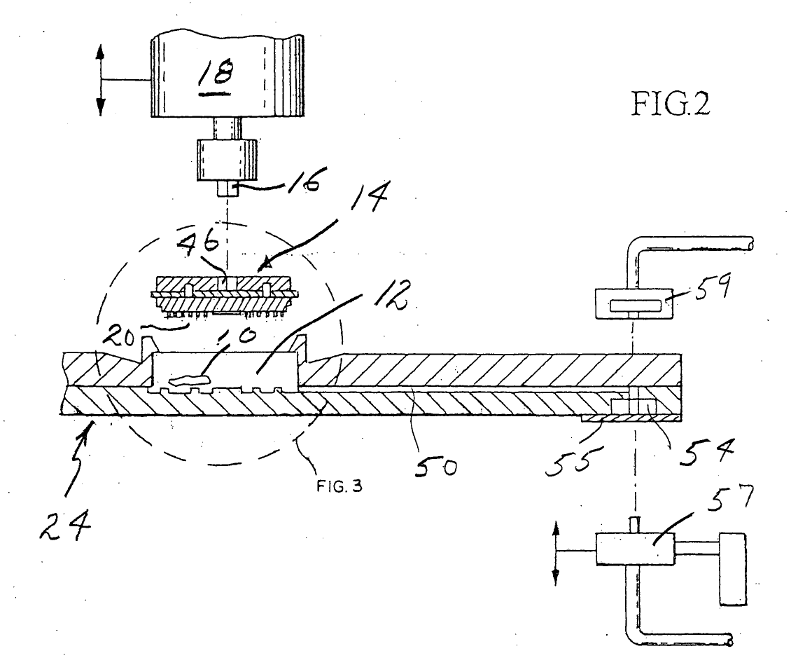

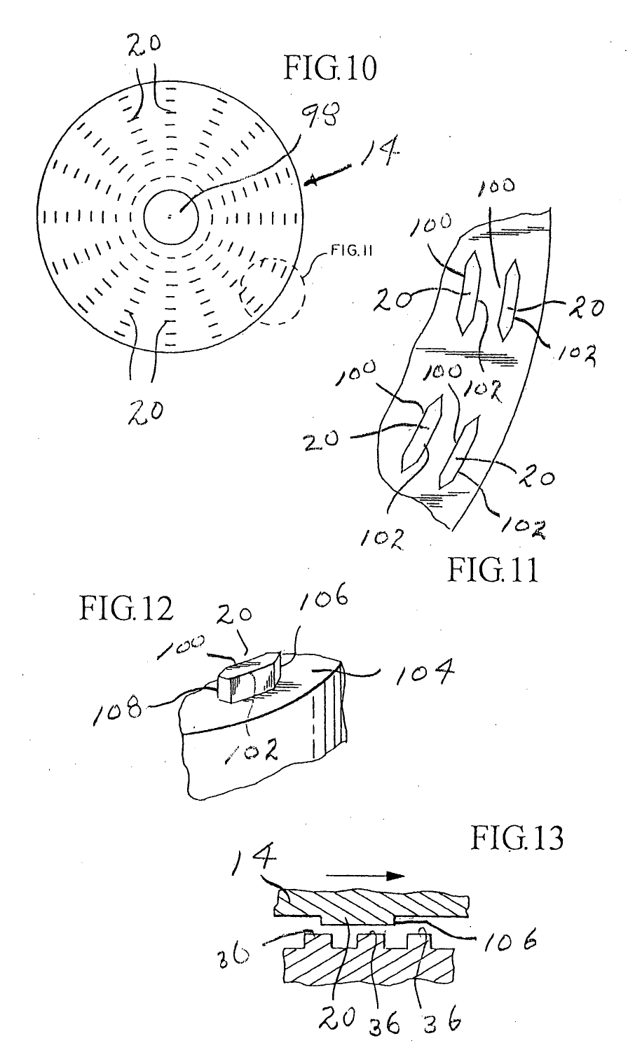

Referring to FIG. 1, a biopsy procedure is performed to obtain some tissue 10 from which cell nuclei are to be removed for testing, e.g., by use of flow cytometry. According to the invention, the biopsy-obtained tissue 10 is placed into a disaggregation chamber 12 which contains a rotor 14. When the tissue 10 is in place, the disaggregator chamber 12 is closed. A drive shaft 16 (FIG. 2) from a motor 18 is connected to the rotor 14. When turned on, the motor 18 rotates the shaft 16 and it in turn rotates the rotor 14. While the rotor 14 is rotating, lytic reagents and stains are introduced into the chamber 12. Mincing edges 20 (FIGS. 10-13) on the rotor 14 mince the tissue 10 until it is divided into very small particles. The mincing edges 20 on the rotor may be approximately 250-2,500 μm in length and 50-1,000 μm in width with ends having about a 2-2,000 μm radius and having height of about 25-200 μm. The radial filtration channels are substantially about 40 microns in width. The rotor, including the mincing edges 20, is preferably cast from a rigid castable urethane plastic or embossed in acrylic. Following this mechanical disaggregation, fluid shear stresses are applied to the tissue 10 to complete cell dissociation in the presence of the lytic reagent. The application of the fluid sheer stress dissociates cells and cell clusters to disrupt cell membranes and release cell nuclei. The released cell nuclei are exposed to nuclear stains and are then directed to a filter that separates the cell nuclei from the connective tissue and debris. The cell nuclei are then delivered to a receiving vessel where they are stored until needed for analysis, e.g., by flow cytometry.

The apparatus of the preferred embodiments comprises housing 24 which may be in the nature of either a one-time-use, disposable cassette, or a reusable cassette 24. FIGS. 2-14 show different constructions of the cassette or housing 24. In each cassette 24, there is a disaggregation chamber 12 and a rotor 14 in the chamber 12.

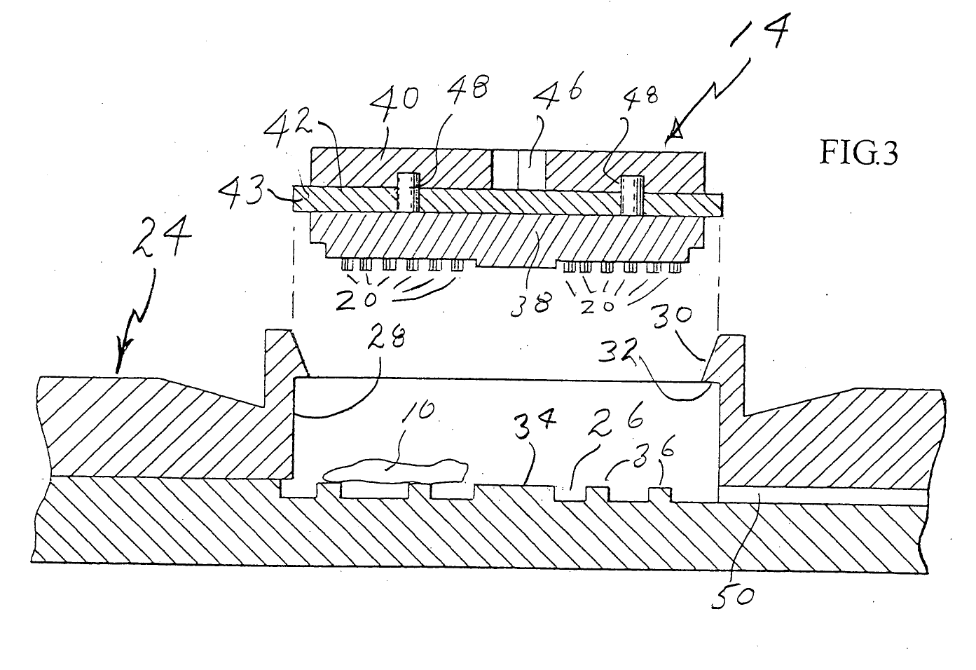

Referring to FIGS. 2 and 3, the chamber 12 may be substantially in the form of a cylindrical socket having a bottom 26 and a cylindrical sidewall 28. The upper part of the sidewall 28 has a conical surface 30 that functions as a rotor latch. An annular radial surface 32 extends between cylindrical surface 28 and conical surface 30 and retains the rotor when inserted. The center 34 of the bottom 26 is preferably flat and is elevated above the bottom 26. A plurality of posts 36 or equivalent features that roughen the surface so as to substantially prevent the tissue from sliding, project upwardly from the bottom 26 and have flat upper surfaces that may be substantially flush with the upper surface of the center member 34 (FIG. 3).

As best shown by FIG. 3, the rotor 14 has an inner (or lower) portion 38, an outer (or upper) portion 40 and a seal member 42 that is sandwiched between the portions 38, 40. Seal member 42 is larger in diameter than the rotor portions 38, 40. As a result, it has an annular outer edge portion that projects radially outwardly, from the diameters of the rotor portions 38, 40, as a “lip” 43. Rotor portion 38 has mincing edges 20 on the side thereof that confronts the floor 26 and the posts 34, 36. The outer rotor portion 40 has a center opening 46 that may be in the shape of a square, a pentagon, a hexagon, an octagon or some other non-circular shape. The flat surfaces of 16 and 40 insure the rotor remain parallel in plane to 24. The seal member 42 is sandwiched between the rotor portions 38, 40 and suitable fasteners 48 are used to connect the members 38, 40, 42 together.

In the embodiment of FIGS. 2 and 3, the biopsy obtained tissue 10 is placed in the chamber 12 and then the rotor 14 is moved into the chamber 12 behind it. The inner rotor portion 38 is placed on the conical surface 30 and is moved downwardly until the projecting “lip” portion 43 of seal 42 becomes positioned below the radial surface 32. The seal 42 has diameter slightly larger than rotor chamber surface 28, thereby creating the sealing action. The rotor shaft and the opening 46 have complementary, non-circular cross-sectional shapes. When the motor 18 is on, it rotates shaft 16 which is connected to the center of the upper rotor part 40 in the center opening 46. When the “lip” 43 is below the surface 32, it contacts and slides along the surface 32 and provides a seal between itself and the surface 32. It also holds the rotor 14 concentric in relation to the chamber surfaces 28 of the housing 24. At that time, the center of rotor portion 38 rest on or is contiguous to the center post 34 and the mincing edges are contiguous to the tops of the outer posts 36.

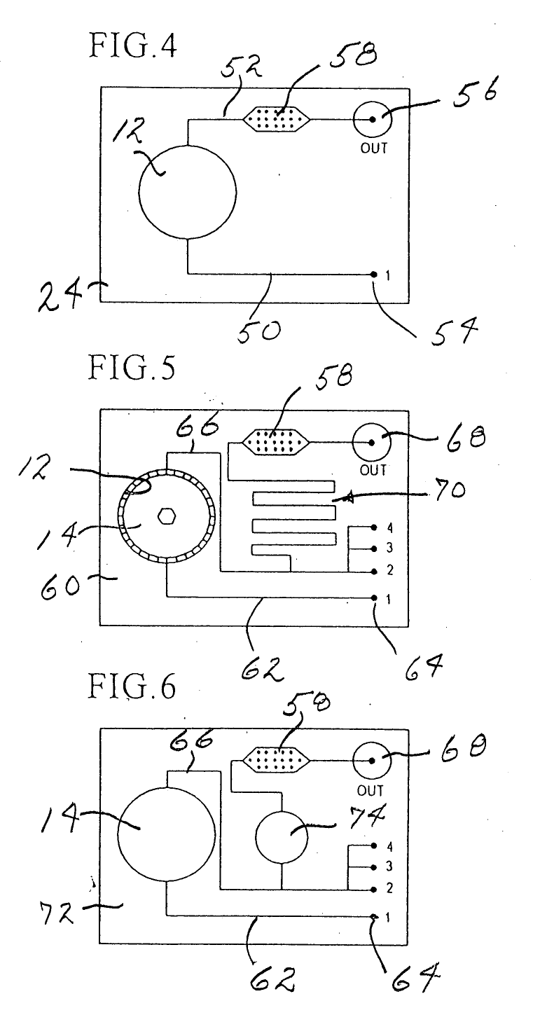

Referring to FIG. 4, the cassette 24 is shown to include an inlet passageway 50 and an outlet passageway 52. Inlet passageway 50 includes an inlet port 54 into which a lytic reagent and stains are introduced. Outlet passageway 52 includes an outlet port 56 for cell nuclei. Outlet passageway 52 also includes a microfabricated filter 58. The inlet port 54 is shown to include a closure in the nature of a patch 55. This patch 55 is connected to the housing about the port 54 by an adhesive. When it is desired to connect the reagent/stain delivery apparatus 57 to the port 54, the patch 55 is merely removed or punctured so as to expose the port 54. Then the delivery apparatus 57 is connected to the port 54. Suitable connector structures are known in the art and for that reason a particular structure is not illustrated. A similar detachable connector structure 59 may be provided for connection to the output port 56.

FIG. 5 shows a second embodiment of the cassette which has been designated 60. It includes a disaggregation chamber 12, an inlet passageway 62, and inlet port 64, an outlet passageway 66, and an outlet port 68. In FIG. 5, the rotor 14 is shown in the chamber 12. The inlet port 64 and inlet passageway 62 are like the port 54 and passageway 50 in FIG. 4. The outlet passageway 66, however, leads to a passive mixer 70 shown as a series of loops in the passageway 66. The loops of the passive mixer 68 lead into a filter 58 that is like filter 58 in the embodiment shown by FIG. 4. Filter 58 leads to outlet port 68 that may be like outlet port 56 in the embodiment of FIG. 4. The embodiment of FIG. 5 includes additional inlet ports, 2, 3, 4 which are for additional stains and/or conditioning reagents.

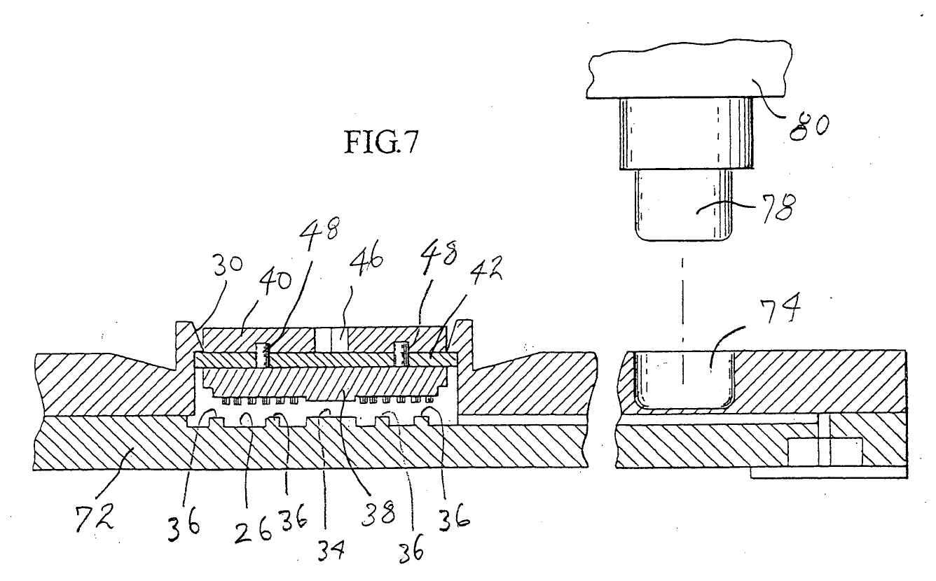

The embodiment of FIG. 6, designated 72, is like the embodiment of FIG. 5 except that it includes an active sonic mixer 74 in place of the passive mixer 70. FIG. 7 is a cross-sectional view taken substantially along line 7-7 of FIG. 6 when the rotor 14 is the chamber 12. It shows a socket 74 in the cassette 72 through which the outlet passageway extends on its way to the filter 58. A sonic exciter 78 is insertable into the socket 74. It is connected to a piezo element 80 which causes the exciter 78 to generate acoustic energy streams within the chamber 74. This results in very efficient mixing of final stage cell lysis.

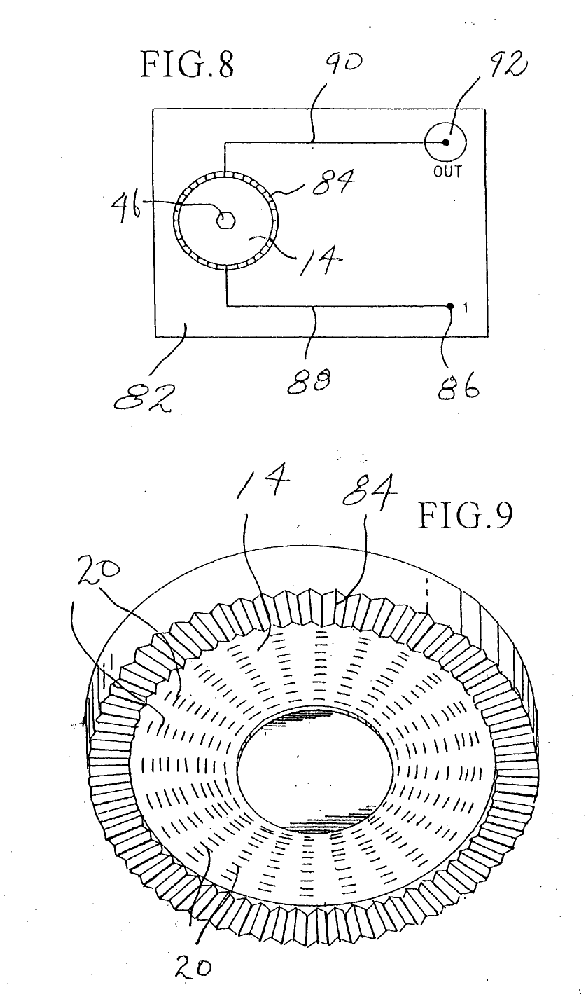

FIG. 8 shows an embodiment 82 that is like the embodiment of FIG. 4 except that in this embodiment the filter is formed by radial channels 84 on the side of the rotor 14 that includes the mincing edges 20. The channels 84 extend radially outwardly from the mincing edges 20 to the periphery of the rotor. Lytic reagent and stain are introduced into inlet port 86 leading into inlet passageway 88 through which they flow into the chamber 12 into contact with the tissue 10. Rotation of the rotor 14 causes the mincing edges 20 to mince the tissue 10, subdividing it into very small particles. These particles then move radially outwardly through the filter channels 84 and ultimately from the filter channels 84 to the outlet passageway 90 that leads to the output port 92.

FIG. 10 a bottom plan view of the rotor shown by FIG. 3. The center region 98 of the rotor 14 is flat and without mincing edges 20. In this embodiment, the mincing edges 20 formed circumferentially spaced apart radial rows of mincing edges 20. The mincing edges 20 are on concentric circles or follow a spiral. They may substantially follow an arc of a circle or a chord of the circle. As shown by FIG. 11, each mincing edge 20 has side edges 100, 102 formed where the bottom surfaces of the members 20 intersect the side surfaces of the members 20. At their opposite ends, the mincing edges 20 converge together to form a blade end. During rotation of the rotor 14, the edges 100, 102 and the leading blade edges all function to cut and mince the tissue 10.

FIG. 12 shows a single mincing edge 20 standing out from a side surface 104 of the rotor 14. In this view, the leading and trailing edges are designated 106 and 108, respectively. FIG. 13 shows the rotor 14 positioned with the mincing edges 20 directed downwardly. The rotor 14 and the mincing edges 20 are shown to rotate relative to the posts 36 formed on the floor of the rotor chamber. It is the movement of the mincing edges 20 relative to the posts 36 that cause the tissue 10 to be cut, minced and reduced in size when the motor is rotating the rotor 14.

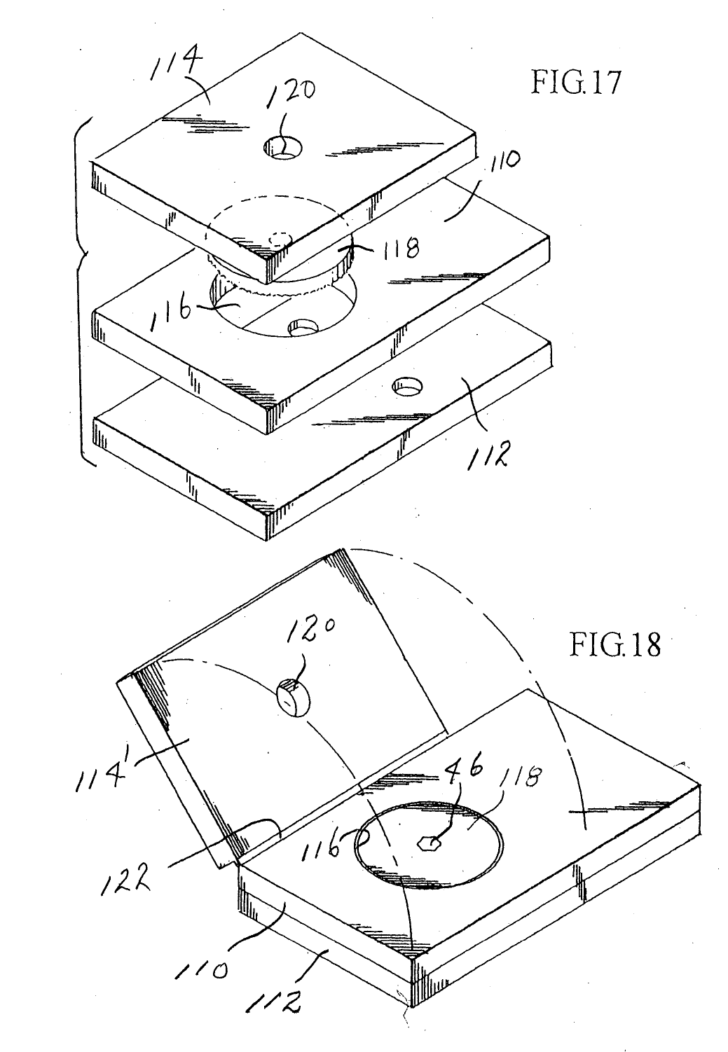

FIG. 17 shows a housing composed of a rotor retainer, a bottom closure 112 and a top closure 114. The rotor retainer 110 includes a rotor-receiving cavity 116 in which a rotor 118 is received. Rotor 118 may be like the rotor shown by FIG. 9, with or without filter channels. The embodiment shown by FIG. 17 may include any one of the filter structures that are disclosed herein. In this embodiment, the top closure 114 is shown to include an opening 120 for receiving a drive shaft from a motor (FIG. 2). FIG. 18 shows the top closure 114′ connected by a hinge 122 to the rest of the cassette housing 110, 112. Hinge 122 permits the closure 114′ to be swung between an open position shown in FIG. 18, and a closed positioned in which it is on the rotor retainer 110. Seals (not shown) may be provided around the opening 120 and around the rotor chamber 116, for sealing against fluid leakage. In this embodiment, the top closure 114′ is swung up into an open position and tissue is placed into the rotor chamber 116. Then, the rotor 118 is placed in the chamber 116 over the tissue. Next, the top closure 114′ is moved into its closed position and in a preferred embodiment a latch secures it closed. Then, the drive shaft 16 is inserted through the opening 120 and into engagement with feature 46.

The top closure 114 can be a separate member, as shown by FIG. 17 and it can be provided with a suitable connector structure (not shown) for connecting it to member 110 when it is desired to close the rotor chamber 116. Many different connector structures can be used for this purpose. Since a particular connector structure is not essential, no particular connector structure is disclosed.

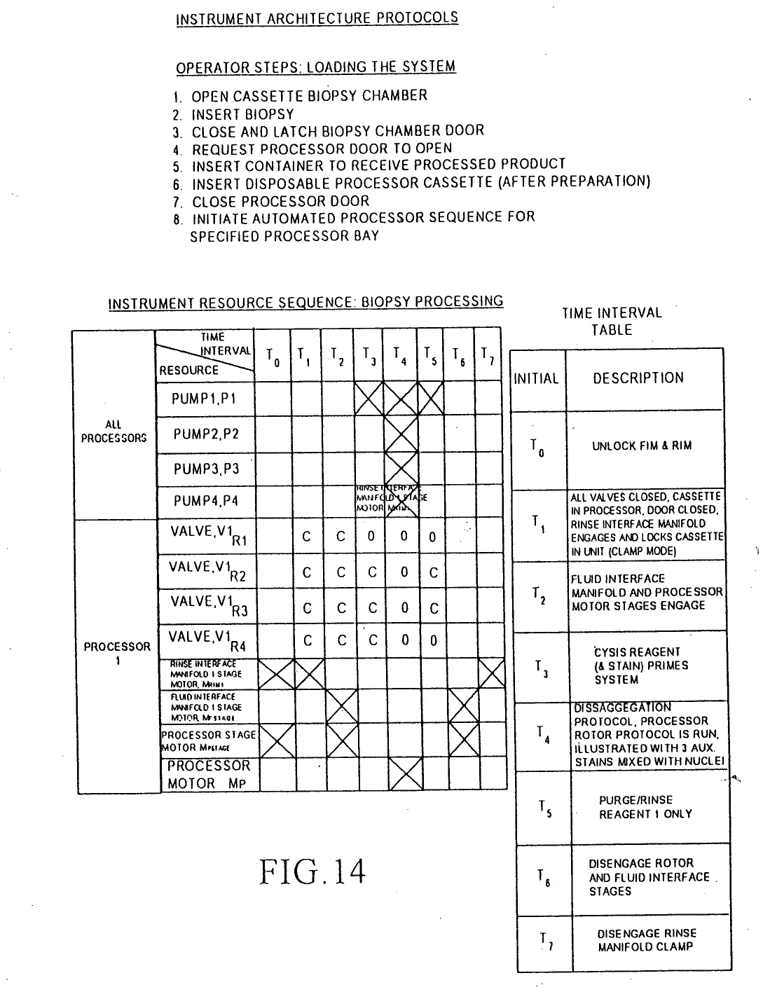

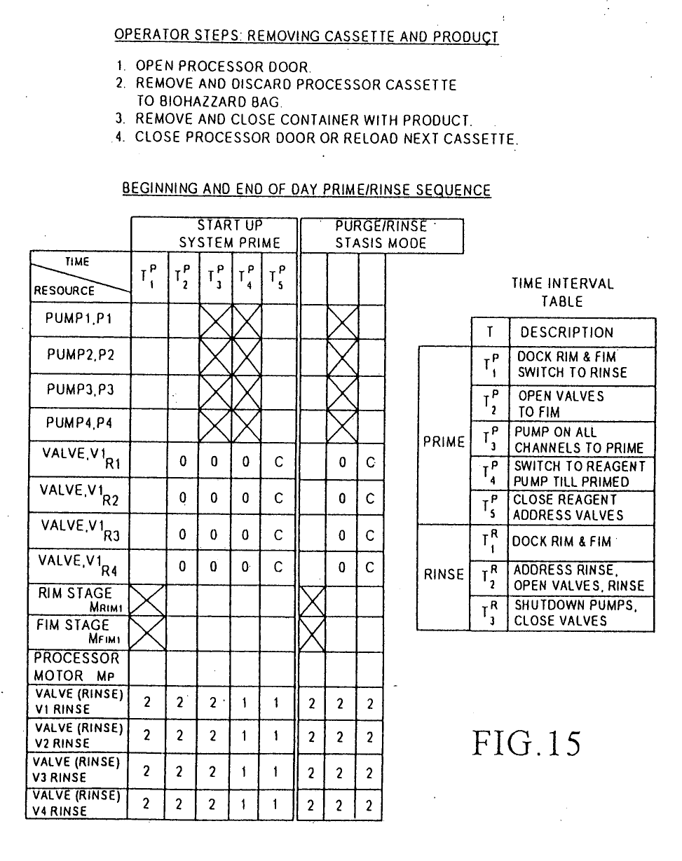

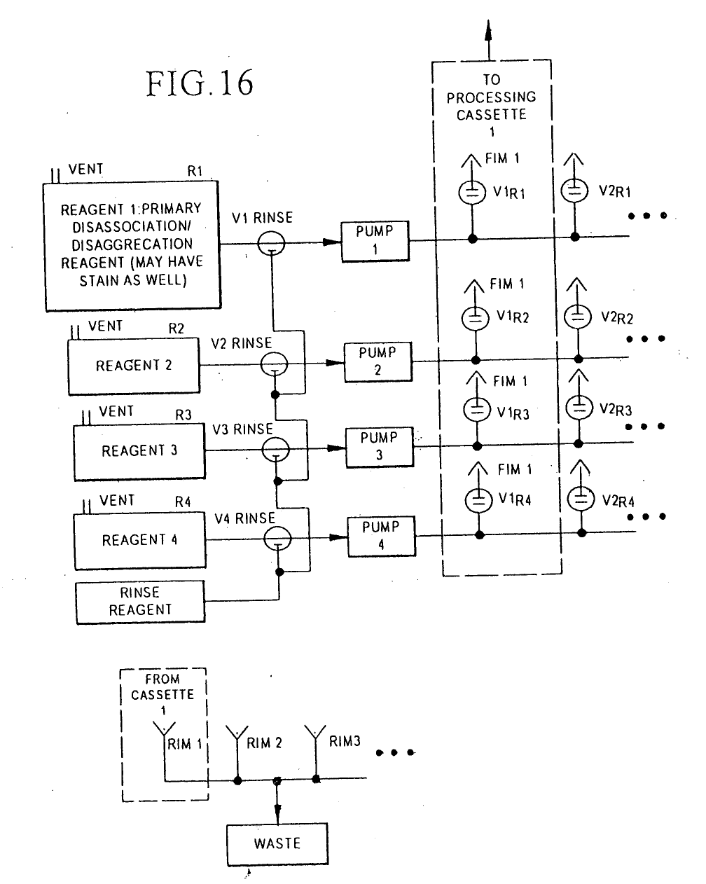

FIG. 16 is a diagram of a system that includes multiple processing stations in parallel with each other. The system may be designed to accommodate one or more, but preferably between five and twenty cassettes simultaneously. Thermal control of all cassettes in Eppendorf tubes will be maintained at 4° C. Reagents would be maintained and chilled thermal blocks at 4° C. It shows reagent/stain delivery to a processing cassette that is at the first processing station. The diagram also shows the cassette connected to waste. The operator steps involved in loading the system and removing the cassette and product from the system are diagramed by FIGS. 14 and 15 and will be described with respect to these figures. In FIGS. 14-16, P1, P2, P3, P4 are pumps. V1, V2, V3, V4 are rinse valves. V1R1, V1R2, V1R3, V1R4 are processing valves. FIG. 14 lists eight operator steps for loading the system. FIG. 15 lists four operator steps for removing the cassette and the product and rinsing out the system.

According to the invention, the biopsy obtained tissue is placed in the disaggregation chamber. The rotor is installed and the chamber is closed. This assembled disaggregation cassette is then placed in a holder in the system. Lysis and staining reagents are added through the reagent/stain inlets. Then, the drive motor is actuated to disaggregate the tissue. The rotational profile of the rotor may be controlled by a computer, thereby enabling complex and repeatable protocols to be achieved. Once disaggregation is completed, a pneumatic pressure may be applied to the inlet port to purge all fluids out from the system. One or more rinse steps are performed on the chamber to recover the maximum quantity of nuclei. In the cassette design as illustrated, the mixing of nuclei staining is performed in the rotor chamber, or in a separate mixer chamber on the cassette with post mixing incubation performed in the receiving Eppendorf tube.

The illustrated embodiments are only examples of the present invention and, therefore, are non-limited. It is to be understood that many changes in the particular structure, materials and features of the invention may be made without departing from the spirit and scope of the invention, therefore, it is our intention that our patent rights not be limited by the particular embodiments illustrated and described herein, but rather are to be determined by the following claims, interpreted according to accepted doctrine of claim interpretation, including use of doctrine of equivalence.

1. A mechanical tissue disaggregator, comprising:
a housing including a rotor chamber;
a rotor in said rotor chamber;
said rotor including a drive-motor interconnect and tissue mincing edges; and
said housing including a tissue inlet to the rotor chamber and the mincing edges on the rotor, and further including an outlet from the rotor chamber.

2. The mechanical tissue disaggregator of claim 1, wherein the housing comprises a rotor cavity in which the rotor is received, and a closure on each side of the rotor cavity.

3. The mechanical tissue disaggregator of claim 2, wherein the drive-motor interconnect is at the center of the rotor and at least one of the closures includes a drive shaft opening that is axially aligned with the drive-motor interconnect of the rotor.

4. The mechanical tissue disaggregator of claim 1, wherein the housing includes a reagent/stain inlet passageway leading into the rotor chamber.

5. The mechanical tissue disaggregator of claim 1, wherein the outlet for the rotor chamber includes an outlet passageway leading from the rotor chamber to an outlet port.

6. The mechanical tissue of disaggregator of claim 1, wherein the mincing edges are spaced apart both radially and circumferentially on a face of the rotor.

7. The mechanical tissue disaggregator of claim 6, wherein the mincing edges are arranged in radial rows on concentric circles.

8. The mechanical tissue disaggregator of claim 1, in which the housing and the rotor are a disposable assembly.

9. The mechanical tissue disaggregator of claim 8, wherein the tissue mincing edges are formed on a face surface of the rotor.

10. The mechanical tissue disaggregator of claim 9, wherein the rotor and the mincing edges are made from a plastic material.

11. The mechanical tissue disaggregator of claim 9, in which the tissue mincing edges are arranged in radial rows.

12. The mechanical tissue disaggregator of claim 12, in which filtration channels are formed on the rotor on the same side as the mincing edges.

13. The mechanical tissue disaggregator of claim 12, wherein the rotor housing includes a reagent/stain inlet passageway leading into the rotor chamber.

14. The mechanical tissue disaggregator of claim 1, wherein the outlet passageway to a microfabricated passive mixing structure for the addition of and mixing of one or more additional reagents with the product stream from the rotor chamber.

15. The mechanical tissue disaggregator of claim 1, wherein the outlet passageway to a microfabricated accoustic streaming mixing structure with external excitation means for the addition of and mixing of one or more additional reagents with the product stream from the rotor chamber.

16. The mechanical tissue disaggregator of claim 8 wherein the housing is made from a plastic material.

17. A method of disaggregating body tissue, obtained by a biopsy, to free and isolate cell nuclei, comprising:
providing a housing that includes a rotor chamber, a rotor in the rotor chamber, and a filter;
providing said rotor with mincing edges;
conducting a biopsy procedure to obtain tissue;
placing the tissue in the rotor chamber contiguous the mincing edges;
rotating the rotor, causing the mincing edges to mince and tease the tissue apart in the pressure of a lytic reagent;
flowing a reagent into and through the rotor chamber for washing disaggregated biopsy tissue away from the mincing edges and carrying it into and through the filter; and removing cell nuclei from the filter.

18. The method of claim 17, providing the chamber with a sidewall portion that is movable between a closed position and an open position;
positioning the side wall portion in the open position to expose the rotor chamber and the rotor in the rotor chamber, then placing the tissue in the rotor chamber contiguous to the mincing edges, and then moving the side wall portion into a closed position in which it extends across and closes the rotor chamber.

19. The method of claim 18, comprising providing a housing and rotor that are a disposable assembly.

20. The method of claim 18, comprising providing a rotor having mincing edges that are made from the rotor mechanical and are positioned on a side surface of the rotor.

21. The method of claim 20, comprising providing tissue mincing edges that are arranged in circumferentially spaced apart radial rows.

22. The method of claim 21, comprising placing the tissue mincing edges on concentric circles.

23. The method of claim 17 having an integrated microfabricated filter.

24. The method of claim 17 having temperature control of the environment in which the processing is performed.

25. The method of claim 17, further comprising applying a pneumatic pressure to the rotor chamber to purge all fluids from the chamber.
